(19) United States
(12) Patent Application Publication
(10) Pub. No.: US 2026/0160748 A1
(43) Pub. Date: Jun. 11, 2026

(54) SYSTEM AND METHOD FOR PREDICTING SOIL ORGANIC CARBON DYNAMICS USING HISTORICAL CLIMATE-BASED MICROBIAL RESPONSE MODELING

(71) Applicant: Perennial Climate Inc., Boulder, CO (US)

(72) Inventors: Mitchell Donovan, Logan, UT (US); Rose Rustowicz, Longmont, CO (US)

(73) Assignee: Perennial Climate Inc., Boulder, CO (US)

(21) Appl. No.: 19/410,176

(22) Filed: Dec. 5, 2025

Related U.S. Application Data
(60) Provisional application No. 63/729,100, filed on Dec. 6, 2024.

(51) Int. Cl. G01N 33/24; G06T 3/4092; G06T 17/05; G06V 10/62; G06V 20/13

(52) U.S. Cl. CPC G01N 33/245; G06T 3/4092; G06T 17/05; G06V 10/62; G06V 20/13

(57) ABSTRACT

The present invention provides a novel solution for predicting soil organic carbon dynamics by combining historical climate data with current ecological indicators through a reduced complexity modeling approach. The method and system of the present invention efficiently predicts where and at what rate carbon sequestration is likely to occur by incorporating a crucial insight: soil microbial communities adapt to local climate conditions over time, creating distinct patterns of carbon sequestration potential across landscapes. The present method and system's ability to track multiple soil health indicators beyond carbon sequestration adds additional value for agricultural and ecological assessment.

### CROSS-REFERENCE TO RELATED APPLICATIONS

This patent document claims priority to earlier filed U.S. Provisional Patent Application Ser. No. 63/729,100, filed Dec. 6, 2024, the entire contents of which are incorporated herein by reference.

### BACKGROUND OF THE INVENTION

The system and method of the present invention relates to the soil organic carbon dynamics prediction industry. In particular, the present invention relates to a reduced complexity biogeochemical model that predicts soil organic carbon dynamics by combining historical climate patterns, current soil, weather, and ecological indicators, and microbial response modeling to determine carbon sequestration potential and soil health indicators across agricultural landscapes at high spatial and temporal resolution. Carbon sequestration is generally known as the process of capturing and storing carbon dioxide (CO₂) to prevent it from entering the atmosphere. It can work through natural methods, as in the context of the present invention, to capture and store the gas in geological formations with the goal of reducing the concentration of greenhouse gases to combat climate change.

By way of background, the following common terms are known in the industry which are pertinent to the description and disclosure of the present invention. For convenience, these terms and their common definitions are provided below.

As to soil science and carbon, the following terms are known. SOC (Soil Organic Carbon) is carbon stored in soil in organic forms, derived from decomposed plant and animal materials. MAOC (Mineral-Associated Organic Carbon) is organic carbon that is chemically bound to soil minerals, representing a stable form of carbon storage. Carbon saturation/saturation capacity is the maximum amount of organic carbon that a soil can stabilize and store, determined by physical and chemical properties of the soil. Pedotransfer functions are mathematical equations that predict difficult-to-measure soil properties from other soil characteristics. Field capacity is the amount the amount of soil moisture remaining after excess water has drained away and the downward movement of water has slowed approximately 1-3 days after a rainfall event. Net Primary Productivity (NPP) is the rate at which plants produce biomass, calculated as the difference between photosynthesis and plant respiration.

As to microbial and biological terms, the following are well known. MMRC (Microbial Moisture Response Coefficient) is a quantitative measure of microbial activity response to soil moisture conditions, based on current and historical climate patterns. MicrobialDegree Days (MDD) is a cumulative measure of time when soil temperatures fall within optimal ranges for microbial activity. Carbon Use Efficiency (CUE) is the ratio of carbon incorporated into microbial biomass to total carbon consumed by microorganisms. Microbial respiration is the release of CO₂ from soil microorganisms as they consume and break down organic matter. Carbon assimilation is the process by which microorganisms convert organic carbon into stable soil organic matter. Anoxic conditions are environmental conditions lacking oxygen, typically occurring in waterlogged soils.

As to modeling and technical terms, the following are well known. RCM (Reduced Complexity Model) is a simplified representation of a complex system that captures essential features while omitting less relevant processes. Biogeochemical (BGC) models are mathematical models that simulate the cycling of elements (like carbon) through biological and geological processes. SMAP (Soil Moisture Active Passive) is a NASA satellite system that provides global measurements of soil moisture. Spatial resolution is the size of the smallest feature that can be detected in a spatial dataset, often expressed in meters. Interpolation methods are mathematical techniques for estimating values between known data points, including a) Bilinear, which is a method using weighted average of four nearest neighbors; and b) Cubic convolution, which is a method using weighted average of sixteen nearest neighbors.

As to agricultural and ecological terms, the following are well known. Rangeland is the uncultivated land that provides forage for grazing animals. Plant Functional Types (PFT) are groups of plant species that share similar ecological roles and responses to environmental conditions. Forbs are herbaceous flowering plants that are not grasses, sedges, or rushes. Lignin content is the amount of complex organic polymers present in plant cell walls that resist decomposition. C:N ratios is the ratio of carbon to nitrogen in organic materials and soils, affecting decomposition rates. Above/belowground biomass is the total mass of living plant material above ground (stems, leaves) and below ground (roots) respectively.

As to data sources and related systems, the following terms are well known. RAP (Rangeland Analysis Platform) is a web-based platform that provides vegetation cover and productivity data for rangelands. PRISM (Parameter-elevation Regressions on Independent Slopes Model) is a climate analysis system that generates spatial climate datasets using point data and digital elevation models. gSSURGO (Gridded Soil Survey Geographic Database) is a digital soil mapping database providing detailed soil information across the United States. Soilgrids is a system for global soil mapping that provides estimates of soil properties at multiple depths.

In the prior art, by way of background, it is well known that estimating the carbon sequestration potential and ecosystem health indicators of agricultural soils at scale represents one of the greatest challenges in developing nature-based climate solutions. The complexity arises from the need to understand and model multiple interacting biological, chemical, and physical processes that occur across vast and varying landscapes. Traditional approaches have struggled to balance accuracy with practical implementation at scale.

In prior art attempts to estimate the rate of carbon sequestration potential and ecosystem health indicators of agricultural soils at scale, practitioners relied on complex biogeochemical models such as DayCent and RothC to predict soil carbon dynamics. While scientifically rigorous, these models were computationally intensive, required extensive input data and calibration, and were primarily developed for cropland systems rather than rangelands. More importantly, they employed static relationships between environmental factors and microbial activity, failing to capture the dynamic nature of soil microbial communities and their evolved adaptation to local conditions over time. Furthermore, the scientific understanding of how soils function and how they sequester carbon has evolved since the introduction of these models.

This limitation in understanding microbial dynamics represented a significant gap in modeling capability. Previous approaches treated microbial activity uniformly across different climate regions, missing the crucial insight that microbial communities evolve and adapt to their local historical conditions. The oversimplified treatment of microbial response to environmental conditions, particularly soil moisture, failed to capture the complex interactions that drive carbon sequestration rates.

As a result, the carbon market faced particular challenges from these limitations. Without efficient methods for “carbon prospecting,” project developers struggled to identify and prioritize lands with the highest sequestration potential. Existing models could estimate total storage capacity but provided little insight into relative sequestration rates across different landscapes or how these rates might change as soils approach carbon saturation. These models often also required site-specific data to make these estimations, which is costly and limits scalability.

Moreover, data integration posed another significant challenge. Available information came from disparate sources-satellite imagery, soil surveys, and climate records-each with different spatial and temporal resolutions. The lack of a unified framework for combining these data streams made it difficult to develop practical, scalable solutions for predicting sequestration potential.

As a result, these limitations created a significant barrier to scaling carbon markets, modeling and understanding soil health, and implementing effective climate solutions. Without the ability to efficiently identify and prioritize high-potential lands, project developers faced increased costs and reduced returns on investment. The lack of tools for predicting relative sequestration rates also made it difficult to optimize resource allocation and maximize carbon sequestration outcomes.

Therefore, there is a fundamental challenge in scaling nature-based climate solutions. There is a need for accurately predicting soil carbon sequestration potential and soil health attributes across vast extents. Due to current low margins in carbon markets compared to high costs, this capability is essential for prioritizing land enrollment in carbon markets and estimating returns on investment, yet traditional approaches have fallen short in several critical ways.

There is a need to be able to capture in a new model how soil microbial communities adapt to local climate conditions over time, instead of relying on static relationships between environmental factors and microbial activity.

There is a further need for these models to be less computationally intensive and require less extensive input data, to make them more practical for rapid, large-scale assessments.

There is yet another need to integrate multiple data streams with different spatial and temporal resolutions, including both historical climate data and current conditions.

These limitations in current methods and systems have created significant barriers to carbon market development. Therefore, there is a particular need for efficient methods for identifying high-potential lands and predicting relative sequestration rates and soil health attributes so that project developers can effectively allocate resources and estimate returns.

Still further, there is a need to account for the effect of carbon saturation on sequestration rates in order to more accurately predict both carbon gains and losses. The complexity of such calculations further compounds these challenges, making it difficult to optimize project planning and investment decisions.

### SUMMARY OF THE INVENTION

Therefore, the present invention provides a new and novel solution for predicting soil organic carbon dynamics by combining historical climate data with current soil and ecological indicators through a reduced complexity modeling approach. The method and system of the present invention efficiently predicts where and at what rate carbon sequestration is likely to occur by incorporating a crucial insight: soil microbial communities adapt to local climate conditions over time, creating distinct patterns of carbon sequestration potential across landscapes. The present method and system's ability to track multiple soil health indicators beyond carbon sequestration potential adds additional value for agricultural and ecological assessment.

The model of the method and system of the present invention accomplishes this by integrating readily available data streams-including, for example, satellite-based (i) soil moisture and (ii) vegetation measurements and (iii) historical climate patterns, to predict microbial response to environmental conditions. The present invention then generates high-resolution maps of sequestration potential and microbial conditions while requiring significantly less computational power than traditional biogeochemical models. The system and method of the present invention's ability to account for how historical climate patterns influence microbial community behavior enables more accurate predictions of carbon sequestration rates and potential across diverse landscapes, which is not heretofore possible in prior art methods and systems. Furthermore, its use of widely available gridded geospatial data results in the ability to produce estimates at global scale without prior farm-specific input data. This is a significant advance over prior art methods and systems.

This solution of the method and system of the present invention provides immediate practical benefits for carbon market development. Thus, project developers can now efficiently identify and prioritize high-potential lands for enrollment in carbon programs, optimizing resource allocation and investment returns. In accordance with the present invention, the model's ability to predict relative sequestration rates, account for carbon saturation effects, and generate multiple soil health indicators provides a comprehensive toolkit for project planning, risk assessment, and identify appropriate management strategies to optimize conditions for SOC sequestration. Additionally, the temporal framework allows for both historical analysis and future scenario modeling, enabling more informed decision-making in carbon project development.

Therefore, there are many differences between the prior art and the method and system of the present invention. As discussed below, prior art methods and systems have many disadvantages and shortcomings compared to the present invention.

For example, the prior art FAO GSOCseq Model uses global soil carbon sequestration modeling, RothC model for predictions, considers vegetation and climate factors, and provides sequestration rate estimates. However, it does not use dynamic microbial response modeling and assumes uniform distributions across plant functional types. This prior art method does not account for historical climate adaptation and lacks microbial activity modeling based on moisture response. This prior art method cannot differentiate sequestration potential between similar vegetation types in different climate regions.

Also, traditional BGC Models (DayCent, RothC) have been used in the prior art, which use comprehensive carbon cycling modeling, consider multiple environmental factors, include microbial processes and use a process-based approach. However, the present invention uses an RCM approach, which requires less computational power while still capturing the core mechanisms involved in soil organic carbon dynamics. The present invention also incorporates historical climate adaptation and dynamics rather than static microbial response curves. Also, the present invention provides integration of modern satellite data streams that is focused on relative sequestration potential rather than absolute rates. As a result, the present invention does not require local on the ground information. Also, the method and system of the present invention can be applied on rangelands and croplands, while prior art BGC models are primarily designed for croplands.

The prior art uses satellite-derived soil moisture data with high spatial resolution and regular temporal updates. On the other hand, the method and system of the present invention uniquely integrates historical climate patterns, employs novel coefficients to capture microbial responses to historical and contemporary conditions (e.g., MMRC, MDD, MpHC, CUE), as well as how these metrics connect and integrate with carbon sequestration modeling.

Microbial models of the prior art fail to consider microbial adaptation to climate and lack a substantive theoretical framework for moisture and temperature responses. On the other hand, the present invention operationalizes the theoretical framework with practical implementation at landscape scale. As with the present invention, integration with multiple data streams is provided along with development of quantitative MMRC, MDD, MpHC, and CUE metrics.

In contrast to previous art, the method and system of the present invention uniquely provides novel integration of historical adaptation with a more sophisticated microbial response modeling. The present invention has inclusion of saturation effects on rates as well as multi-attribute output generation.

Unlike the prior art attempts, the results of the method and system of the present invention achieves a more efficient, scalable approach to “carbon prospecting” that reduces barriers to entry for carbon markets while improving the likelihood of project success, where efficiency is defined as the absence of required farm-specific data, where data availability is for the whole globe, and where it is possible to extract pre-computed results for any field or ranch. This advancement directly addresses the need for practical tools to scale nature-based climate solutions across agricultural landscapes.

Therefore, an object of the present invention is to combine historical climate adaptation with current conditions for microbial response prediction. The invention does so through the use of a mathematical framework of nested computations that continuously adjust modelled microbial responses to contemporary conditions based on historical climate regimes.

Another object of the present invention is to provide efficient integration of multiple data streams while also maintaining sufficiently fine spatial resolution to enable decision making.

Yet another object of the present invention is to account for dynamic microbial responses to moisture and cumulative time spent within optimal soil temperatures.

A further object of the present invention is to effectively differentiate sequestration potential across similar landscapes with different climate histories.

### BRIEF DESCRIPTION OF THE DRAWING FIGURES

Further advantages, features and possible applications of the present invention are shown and described in the accompanying drawing figures.

FIG. 1 is a flow chart showing the microbial access constraints, microbial metabolic limitations (MML), sequestration potential projections and soil carbon saturation features of the method and system of the present invention; and

FIG. 2 shows further details of the Carbon Use Efficiency (CUE) ratio of carbon incorporated into microbial biomass to total carbon consumed by microorganisms.

FIG. 3 shows further details regarding soil temperature in the form of a graph of respiration rate compared to temperature.

FIG. 4 shows further details regarding soil pH in the form of a graph of phylotype richness compared to soil pH.

FIG. 5 shows further details regarding soil moisture in the form a graph of microbial activity compared to soil moisture.

FIG. 6 shows a sample map of soil organic relative potential sequestration in a portion of the United States modeled in accordance with the present invention.

FIG. 7 is a close-up view at local-scale spatial variability in accordance with the present invention and following the same gradient as seen in FIG. 6.

FIG. 8 is high-resolution visual imagery of the image of FIG. 7 and following the same gradient as seen in FIG. 6.

FIG. 9 is a chart showing variability in SOC sequestration potential across states and dominant plant functional types.

FIG. 10 is a graph showing distribution of relative sequestration potential for eight US states across the western United States.

FIG. 11A is a comparison between the method results of the method of the present invention and the prior art FAO GSOCSeq modeling method for the perennial forb/grass plant functional type as aligned with distributions from the literature.

FIG. 11B is a comparison between the method results of the method of the present invention and the prior art FAO GSOCSeq modeling method for the perennial tree plant functional type.

FIG. 11C is a comparison between the method results of the method of the present invention and the prior art FAO GSOCSeq modeling method for the perennial shrub plant functional type.

### DESCRIPTION OF THE INVENTION

As further shown in the attached figures, the system and method 10 of the present invention is shown and described in detail.

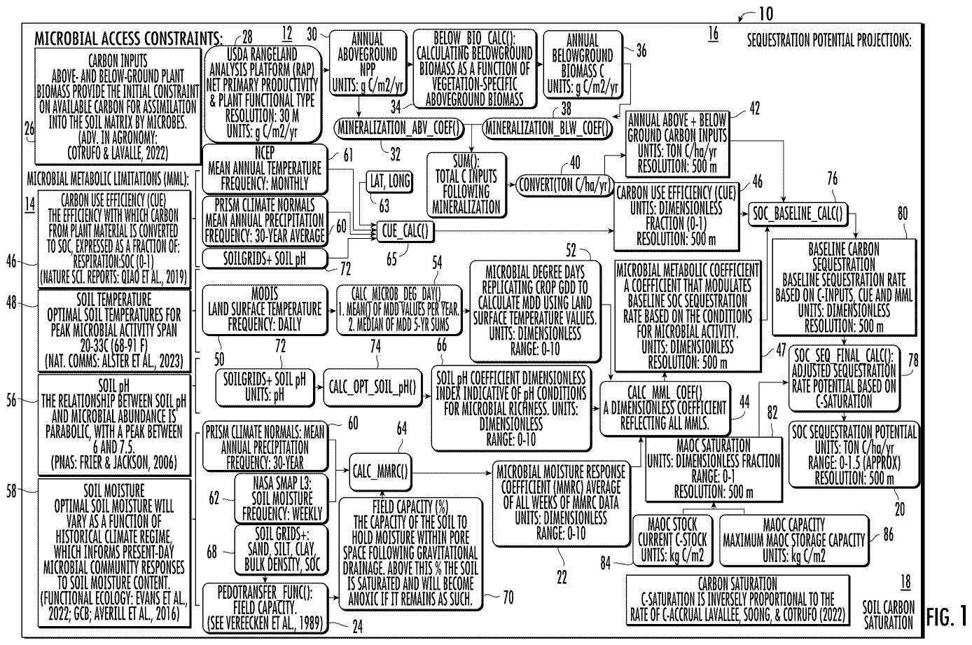

Referring to FIGS. 1, the method and system 10 of the present invention is shown in detail. The present invention 10 employs a reduced complexity biogeochemical modeling framework that predicts soil organic carbon dynamics by synthesizing multiple data streams into a cohesive analysis of microbial activity and carbon sequestration potential. At its core, the system leverages the novel insight that microbial communities adapt to historical climate conditions, creating distinct patterns of carbon processing efficiency across landscapes.

More specifically, referring first to FIG. 1, the model of the method and system 10 of the present invention is shown in detail to include the features of: a) microbial access constraints 12, b) microbial metabolic limitations (MML) 14; c) sequestration potential projections 16, and d) soil carbon saturation 18 to arrive at a SOC Sequestration Potential 20, as will be described in detail below.

In general, the method and system 10 of the present invention operates by first establishing functions that continuously adjust as a function of historical climate conditions (i.e., 30-year precipitation norms) to determine the most representative metabolic response curve of local microbial communities. The present model 10, using continuous functions rather than classes, operates by first applying novel microbial-transfer functions to historical climate data (e.g., 30-year precipitation averages) to determine the characteristic response of local microbial communities. The transfer function is a continuous bell-shaped curve that adjusts with the long-term mean annual precipitation. The function is then combined with current satellite-derived soil moisture measurements, soil physical structure, and soil chemical properties, to calculate a Microbial Moisture Response Coefficient (MMRC) 22. Adapted communities are “adapted microbial communities”. Microbial communities in soils adapt (evolve) to adjust to the conditions present in their soils. If the conditions are not favorable for a specific community, that community will shift or completely die out to be replaced by a community that is able to survive there. This coefficient 22 reflects how these adapted communities will respond to present conditions. The method and system 10 accounts for soil physical properties through pedotransfer functions 24 to estimate field capacity and other crucial soil chemistry characteristics that influence microbial activity. More specifically, a pedotransfer function 24 is an equation that takes in very simple, easy-to-measure, properties of soils and uses those to calculate complex, hard-to-measure, properties of soil. In accordance with the present invention, a pedotransfer function 24 is used to calculate field capacity, which is the ability for a soil to absorb and hold onto water following a rainfall event. This is calculated because it impacts soil moisture conditions, which is an aspect of MMRC 22 calculations of the present invention 10 and indicated as f_{c} in the equation discussed below.

First, the microbial access constraints are considered, such as carbon inputs 26, which are above-ground and below-ground plant biomass to provide the initial constraint on available carbon for assimilation into the soil matrix by microbes. USDA Rangleland Analysis Platform (RAP) at 28 is also employed to factor in New Primary Productivity and Plant Functional Type, using a resolution of 30 m with the units of g C/m2/yr. Thus, the Annual aboveground NPP at 30 is be determined with the units of g C/m2/yr to generate the coefficient of mineralization_abv_coef( ) 32. The belowground biomass 34 as a function of vegetation-specific aboveground biomass is determined as below_bio_calc( ) to provide an Annual belowground biomass C at 36, with the units of g C/m2/yr to generate the coefficient of mineralization blw_coef( ) 38. The coefficients of mineralization_abv_coef( ) and mineralization_blw_coef( ) are totaled to provide a value of convert (ton C/ha/yr) at 40 to, in turn, provide as part of the sequestration potential projection at 16 as the Annual above+below ground carbon inputs with the units of C/ha/yr with a resolution of 500 m at 42.

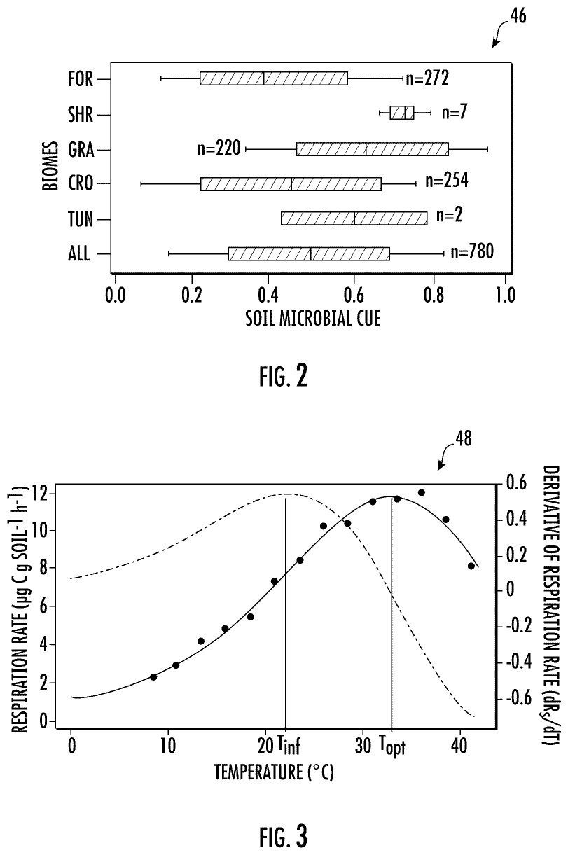

Still referring to FIG. 1, the microbial metabolic limitations (MML) at 44 are also shown to arrive at a microbial metabolic coefficient 47 and how this feature contributes to the sequestration potential projection 16. The Carbon Use Efficiency 46 is used, which is the efficiency with which carbon from plant material is converted to SOC, expressed as a fraction of respiration: soc (0-1). The graph of FIG. 2 shows further details of Biomes compared to Soil Microbial CUE in connection with the noted carbon inputs, such as found from the publication entitled “Global variation of soil microbial carbon-use efficiency in relation to growth temperature and substrate supply”, Qiao et al, 2019, Sci Rep 9, 5621.

Soil temperature 48 is also a factor in the method and system of the present invention 10, which is the optimal spoil temperatures for peak microbial activity across the span of temperatures 20-33° C. (68-91° F.), which uses MODIS satellite imagery 50 to determine land surface temperature for the novel calculation to the field of biogeochemistry, which is MicrobialDegree Days (MDD) 52 in accordance with the model of the present invention. FIG. 3 shows sample data for respiration rate compared to temperature for use with this module 48 of the present invention 10, such as that found in publication entitled “Quantifying thermal adaptation of soil microbial respiration”, Alster et al., 2023, Nat Commun 14, 5459.

As to the MicrobialDegree Days (MDD) Assessment at 52, the method of the present invention 10 downloads 8-day land surface temperature data for all areas with land surface temperature data available from the MODIS satellite (or an equivalent satellite with thermal measurements) 50. Land surface temperature data is incorporated with known temperature ranges within which microbes function optimally to calculate a score (0-10) for each 8-day window. The score 54 is calculated using a novel function that is highest when temperatures are most frequently within the optimal temperature range that is derived from empirical measurements in literature.

As part of the MDD calculation 52, crop GDD is replicated to calculate MDD using land surface temperature values. As a result, the MDD 52 has normalized scores in the range of 0-10 with dimensionless units. The MDD 52 has a formula structure of:

$$MDD = mean ( daily\_temp \_response \_factor )$$

For further background, regarding MicrobialDegree Days (MDD) 52, microbial communities evolve and adapt to different temperature optima based on historical thermal regimes. An analogous relationship exist for crops that have adapted to certain climates, where a range of air temperatures (e.g., 50-80 degrees F.) are known to promote maximal growth for crops, and temperatures outside of this range inhibit or even degrade crop growth. The accumulated time that is spent within this window is quantified by a metric called GrowingDegree Days (GDD), also at 52. Herein, the present invention 10 adapts these broadly accepted and useful concepts to quantify a novel metric: MicrobialDegree Days (MDD) 52, wherein the same formulation of GDD to soil temperatures that are most suited to microbial activity. The MDD 52 formulae are applied over 5 years to calculate the 5-year average MDD using 8-day land surface temperature values derived from MODIS 50, as discussed herein. The use of 5-years ensures that the range of plausible and recent conditions for each land parcel are accounted for, reducing the likelihood of biases arising due to outliers if too few years were used, while avoiding historical data that may be irrelevant to present temperature regimes. The method 10 of the present invention assumes a temperature range spanning those found in literature are the optimal ranges for both microbial activity and microbial CUE 46. Soil temperature data 48 spanning 5 years to present is used to quantify typical conditions and avoid any single anomalous year which could be skewed due to anomalous conditions. In doing so, the ability to project future trends are based on the central tendency of historical temperature conditions. The outputs are then normalized to a range of 0-10 to ensure equal weighting. Thus, the present invention employs novel temporal aspects as part of a soil sequestration analysis.

This is a novel transfer of GrowingDegree Days (GDD) 52 from agronomic forecasting that the current art uses to account for the effects of soil temperature on microbial activity and adjusts sequestration predictions based on current carbon saturation levels. The calculations underpinning MDD 52 scale monotonically with the cumulative time a soil spends within the range of soil temperatures known to facilitate optimal microbial activity. In accordance with the present invention, the product of these components (MMRC 22 and MDD 52), along with the soil pH scalar 56, are used to generate a dimensionless sequestration coefficient, namely, calc_MML_coef( ) 44, which ranges from 0 to 10. This coefficient 44 is used to scale carbon inputs from then vegetation, which is further modulated by carbon saturation levels, namely, to generate comprehensive predictions of sequestration potential. The dimensionless and dimensional variables are combined mathematically so that their product produces model outputs in units typical of biogeochemical models (mass of carbon per unit area per unit time).

By processing this information at high spatial resolution (30 m-500 m) and annual temporal frequency (and resolution), the method and system 10 generates detailed maps (i.e., raster outputs that are provided to users indirectly through a graphical user interface or directly as a .tif or .pdf) of relative sequestration potential across landscapes, see FIGS. 6-8 discussed below. While raw remote sensing data straight from satellites can be at 10-30 m, it is uncommon for subsequent/derivative data products to be produced globally at such high resolution, often because the computational burden is quite high. This is especially true for models, which rely on many inputs, and thus, are even less likely to be at high resolution. The invention 10 is based on all of the highest resolution products available and for those that were at coarser resolution, they are preferably upscaled to higher resolutions for use in accordance with the present invention 10. While finer spatial resolutions may result in better capturing the spatial heterogeneity (i.e., spatial variability), there is a trade off with data size and processing speed.

As a result, the reduced complexity approach of the model 10 of the present invention enables efficient batch system processing of large geographical areas while maintaining accuracy in predicting relative sequestration rates. This framework allows for both historical analysis and future scenario modeling, providing a practical tool for tracking historical conditions, developing carbon projects, annual soil health monitoring, and forward-looking sequestration projections. Such efficiency is not possible in prior art methods and systems.

As to the technical process of the present invention, the method and system operates through several other interconnected technical processes and components, namely the factors of soil pH 56 and soil moisture 58.

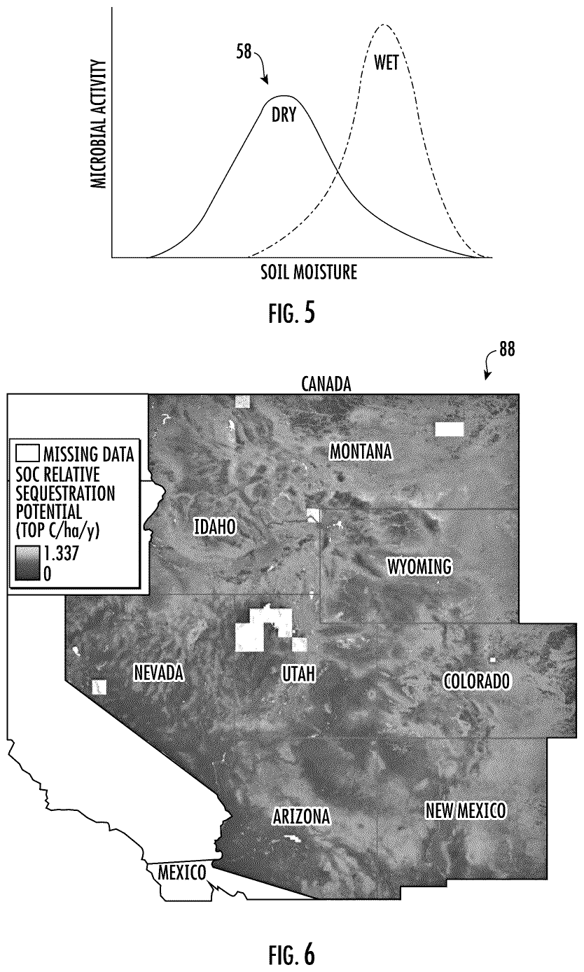

Referring first to the soil moisture data as part of the method and system of the present invention at 58, where a historical climate analysis component is provided. FIG. 5 shows a graph of microbial activity compared to soil moisture to further illustrate this component of the present invention.

This historical climate analysis component includes the processing of 30-year precipitation data (e.g., the PRISM Climate Group at Oregon State University at 60) using continuous microbial-transfer functions to generate a microbial activity response curve. Each unique response curve reflects the range of possible microbial responses that may ensue for any plausible contemporary soil moisture conditions, where different curves are derived based on the 30-year historical climate data. The functions used are novel (first applied by the present invention) based on conceptual/theoretical frameworks established in biogeochemical research. The continuous functions used to generate the response curves were chosen to reflect the most recent understanding of how different microbial communities respond to the same contemporary conditions due to historical adaptations. This data processing establishes the baseline metabolic activity response curve of the microbial community at a given location based on the 30-year average precipitation long-term, and does not preclude the use of shorter or longer historical averages. The response curves are in the form of Gaussian distributions that adjust based on the value of the 30-year mean annual precipitation for each pixel. A pixel represents the minimum spatial area over which the computations of the model are generated/represented, where multiple pixels make up a map that is the continuous non-homogenous representation of a broader spatial area. The equation represents the relative rate of microbial activity in response to specific moisture levels if all other variables are held constant. For example, soil microbial communities that have adapted for survival within arid environments will differ in their response to a soil at 20% moisture content as compared to a soil microbial community within a tropical region. Further soil moisture data from NASA SMAP L3, at 62, with a weekly frequency, is used with the PRISM data 60 to arrive at the value calc_mmrc at 64.

A Microbial Response Modeling Framework is provided in accordance with the present invention where a Microbial Moisture Response Coefficient (MMRC) 22 is calculated. The SMAP satellite soil moisture data 58 is incorporated into the response-curve functions generated as part of a Historical Climate Analysis Component. The mathematical structure allows modeling as to how microbial community activity will respond to present-day soil moisture properties. These values are summarized over one- and five-year time periods. This outputs daily/weekly moisture response coefficients. This process generates a geo-referenced grid of values at the target resolution. Thus, the formula structure for the MMRC coefficient 22 is:

$$MMRC = f ( current\_moisture , historical\_climate , field\_capacity )$$

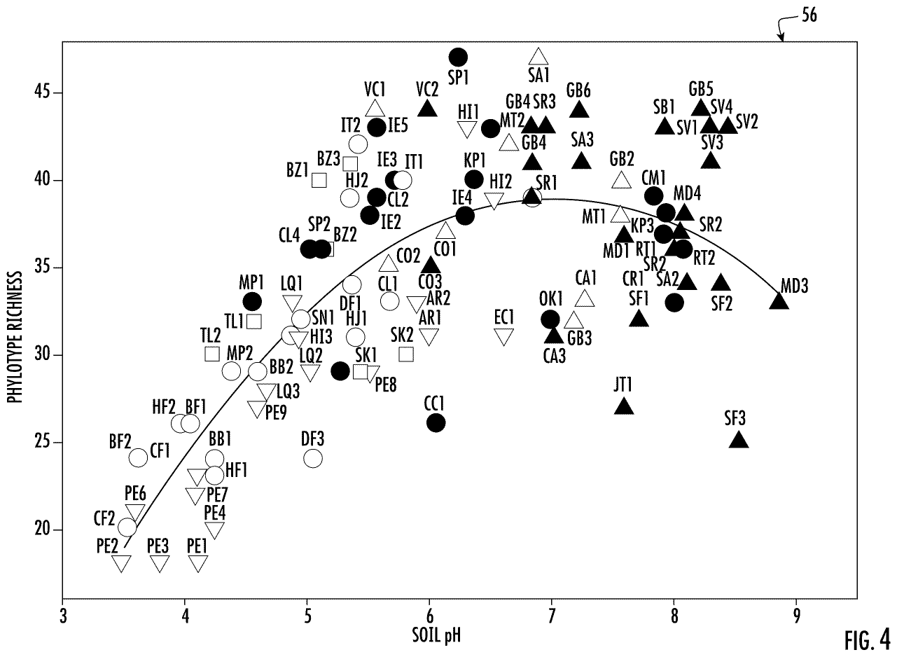

Also as part of the soil physical properties module is the relationship between soil pH 56 and microbial abundance being parabolic, with a peak between 6 and 7.5. FIG. 4 is a graph showing phylotype richness compared to soil pH 56, such as found from the publication entitled “The diversity and biogeography of soil bacterial communities”, Fierer et al, 2006, Proc. Natl. Acad. Sci. U.S.A. 103, 626-631. Such soil pH data 56 is used based on data to calculate a dimensionless variable (0-1) that captures how favorable the pH is for enabling or inhibiting microbial activity. Thus, a soil pH coefficient 66 is determined, via calculation calc_opt_soil-pH( ) 74, to provide a soil favorability rating, where higher values indicate more favorable pH values for soil microbial communities to process and assimilate carbon into the soil. The equation below captures the shape of the relationship between soil pH (derived from gridded soil data) and the efficiency of microbial activity across the range of plausible soil pH values, where microbes are inhibited at extremely acidic and alkaline soils, but optimized at a central pH derived from literature.

$$\frac{10}{( \sqrt{2 π {s}^{2}} )} exp ( - \frac{{( x - m )}^{2}}{12 {s}^{2}} )$$

Where x reflects the soil pH value for a given location, s is a scalar value currently tuned and set to 0.4, and m is a scalar currently set to the pH that corresponds with peak microbial activity (i.e., 6). While these are currently set, leaving them as variables allows future refinements to capture adjusted values for a range of alternative conditions.

This allows the quantification of how the range of alkaline to acidic soils can lead to more or less favorable conditions for microbial activity, as it relates to carbon sequestration. The value varies from 0-10 but it could be adjusted to vary from 0-1 by dividing by 10.

This Soil Physical Properties Module implements pedotransfer functions (PTF) 24, which are predictive functions of certain soil properties using data from soil surveys, using soil texture data. This module calculates field capacity 70 using equations derived from literature that incorporate soil physical and chemical properties (e.g., see below) from SoilGrids and SoilGrids+ 68. The outputs are in absolute units (%) from: % sand, silt, clay, organic matter content, and bulk density. Its formula structure is as follows:

$$field\_capacity = f ( texture , OM , bulk\_density )$$

As part of the soil physical properties module is a Soil Chemical Properties Module that uses soil pH inputs from SoilGrids+ at 70 within a novel microbial transfer function to estimate a dimensionless coefficient that scales with microbial activity responses to soil pH (referred to as MpHC). As noted above, the response function is parabolic and reflects empirical data found across multiple literature publications.

In accordance with the present invention, the aforementioned modules and calculations (MMRC, MDD, MpHC) are used to calculate a relative/dimensionless sequestration score. The dimensionless sequestration score is calculated as the mean of the MMRC, MDD, and MpHC values, as follows:

$$seq\_score = f ( MDD , MpHC , MMRC )$$

Subsequently, the sequestration score is multiplied by carbon use efficiency (CUE) 46 and the incoming carbon from above and belowground carbon inputs to establish a baseline sequestration rate potential value soc_baseline_calc( ) 76 for a baseline carbon sequestration rate 80. The computation uniquely incorporates novel input datasets (e.g., MMRC, MpHC, and MDD) to establish a baseline carbon sequestration rate potential or soc_seq_final_calc( ) 78.

A carbon saturation adjustment is made where the baseline carbon sequestration potential 78 is adjusted based on the saturation (%) of mineral-associated organic carbon content that is currently present at a given location. Carbon saturation is inversely proportional to the rate of C-accrual. The saturation data is derived from a publicly-available dataset published in Nature. The MAOC Saturation 82 is a dimensionless fraction with a range of 0-1 at a resolution of 500 mm from data from MAOC Stock (units of kg C/m2) 84 and MAOC Capacity 86, which is maximum MAOC storage capacity (units kg C/m2).

The method also includes NPP integration where aboveground Net Primary Productivity from MODIS 50 is used alongside allometric equations from literature to calculate belowground biomass, and from there derive total incoming carbon into the soil matrix. The average of 5-years (but could be one year or two years or three years or four years) is used to avoid making long-term projections based on a single year of (potentially) anomalous data and to account for crop/vegetation rotations, where annual data is more useful for short-term understanding of carbon dynamics, but does not enable projection long term because any given year doesn't represent the “typical” conditions. Whereas 5 year periods give a better indication of what range of conditions exist for a given location.

The Final Sequestration Potential 20 is determined based on the following formula:

=baseline_adj_seq_rate×seq_score×saturation_adjustment_factor

where the baseline_adj_seq_rate=f(biomass, CUE), where biomass=f(NPP, land cover types). Also, the aboveground biomass is derived from typically raw MODIS Satellite data 50. This is combined with functions unique to each plant functional group to derive belowground biomass. CUE 46 is derived using geographic location (Lat/Long) 63, Mean Annual Precipitation, (NCEP) Mean Annual Temperature 61, and soil pH where cue_calc( ) 65 is carried out.

The present invention 10 further includes a temporal Analysis Component with Annual frequency processing, preferably annual/1 year frequency for temporal resolution. The frequency of availability, meaning that data is offered at e.g. 1-year frequency (temporal resolution). Historical analysis capability is provided, where the framework is to generate estimates of the described properties every 1 year for the past 20-30 years, allowing the construction of a time series that allows for comparison of the soil health/carbon trends of land parcels.

For future scenario modeling, instead of taking the input data from remote sensing, scenarios of input data is created based on different scenarios of what might happen in the future. So for example, instead of taking observed net primary productivity (plant productivity), a model could be created to predict what would happen if plant productivity decreased by 1% every year for the next 10 years. This results in the prediction of carbon gains and losses.

It should be noted that FIG. 1 represents an algorithm and model for the USA by way of example in accordance with the present invention. While there may be some differences in data sources and methodology for the global version, the process for such use and application of the present invention is the same and is considered within the scope of the present invention.

Referring now to FIGS. 6-11, sample data results in accordance with the method and system of the present invention is shown.

FIG. 6 shows a sample map 88 of soil organic relative potential sequestration in a portion of the United States modeled at 500 m resolution in accordance with the present invention. The data is expressed as annual projected sequestration rates based on actual carbon inputs where the values are interpreted as relative sequestration rates. Such resolution and accuracy of SOC sequestration rate potentials have never been modeled at broad scales, and models that could attempt to do so have not included microbial adaptations into their model architecture.

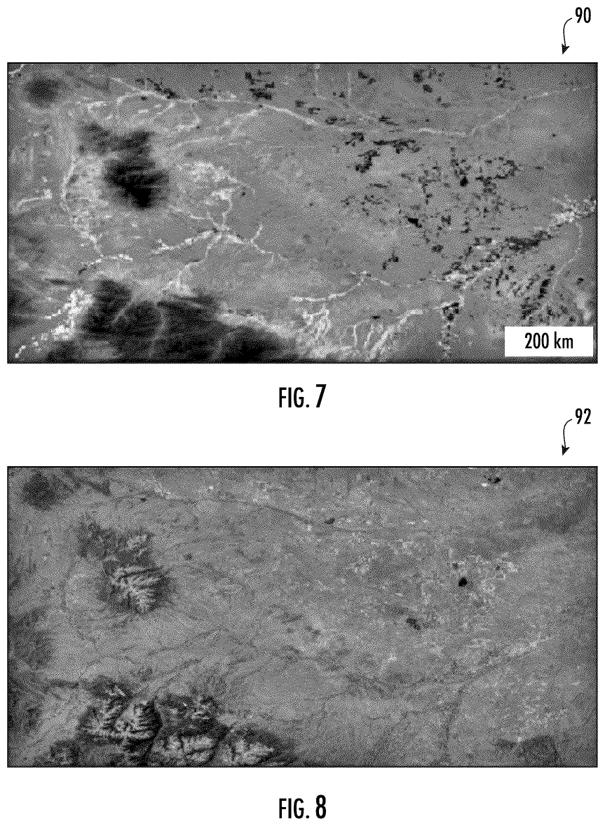

FIG. 7 is a close-up view 90 at local-scale spatial variability in accordance with the present invention and following the same gradient as seen in FIG. 6. FIG. 7 demonstrates incredible alignment with topographic, hydrologic, geomorphic, and vegetation/biomass dynamics found in the visual imagery (FIG. 8) for the exact area of interest. This is a clear demonstration of the improvement over past efforts to model spatial variability in SOC sequestration rate projections. FIG. 8 is high-resolution visual imagery 92 of the image of FIG. 7 and following the same gradient as seen in FIG. 6. Each map is 200×120 km.

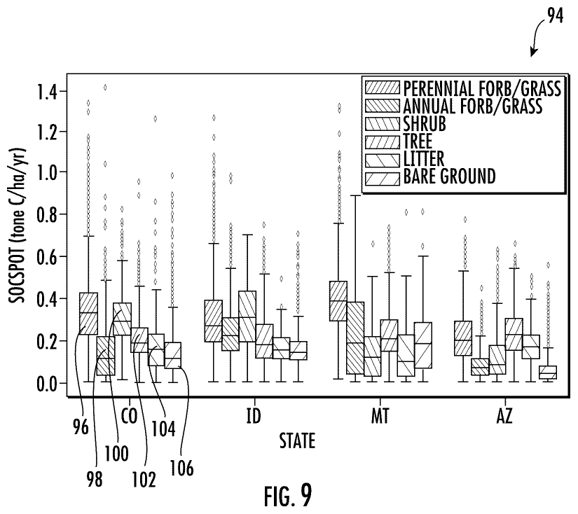

FIG. 9 is a chart showing variability in SOC sequestration potential across states and dominant plant functional types 94, namely perennial forb/grass 96, annual forb/grass 98, shrub 100, tree 102, litter 104, and bare ground 106. The data for each plant functional type is in the same order left to right for each state. Generally, as can be seen, areas with predominantly perennial forb/grasses exhibit the greatest potential for SOC sequestration. However, where sufficient microbial metabolic limitations (MML) exist, land with dominated by other PFTs can meet or exceed perennial grasses (e.g., Idaho annual grasses and shrubs). The designation PFTs are based on the majority of coverage within a 500 m pixel, and should not be interpretated as sole vegetation. Data from each state reflects 10,000 points randomly generated and sampled across the extent of the state.

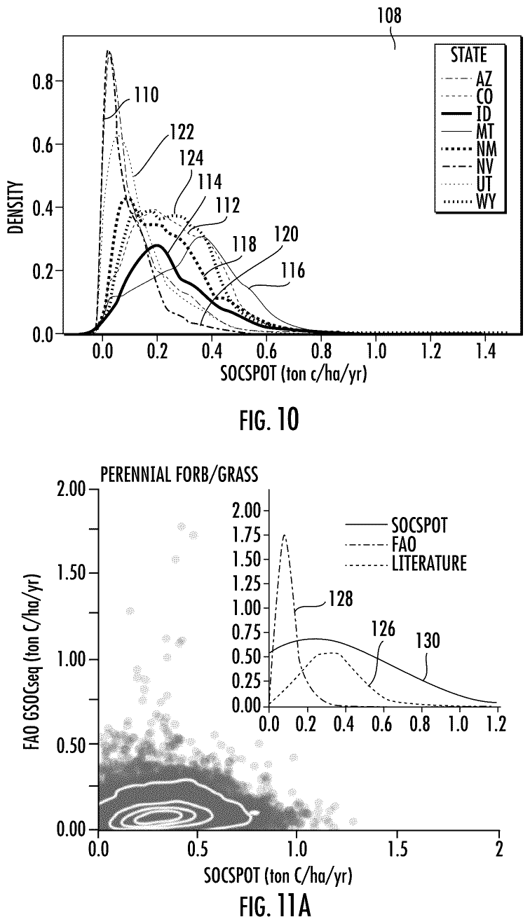

FIG. 10 is a graph 108 showing distribution of relative sequestration potential for eight US states across the western United States including Arizona 110, Colorado 112, Idaho 114, Montana 116, New Mexico 118, Nevada 120, Utah 122 and Wyoming 124. For example, drier states narrower growing seasons (e.g. Arizona and Nevada) have significantly lower mean sequestration rates compared to states with favorable climates (e.g., such as Montana and Idaho) for microbial activity.

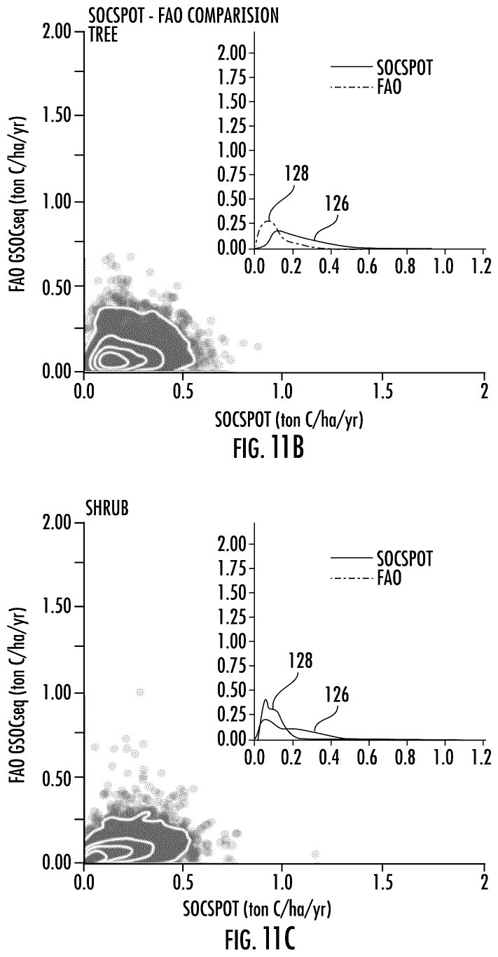

FIGS. 11A-C show various comparisons between the method and system of the present invention and the prior FAO GSOCSeq model for three dominant plant functional types where no significant alignment was indicated. Within the subset plot found in FIG. 11A, the alignment between the histograms for SOCSPOT 126 and literature 130 demonstrates clear improvement relative to the previous efforts of FAO GSOCSeq 128 plotted alongside. Further, the variability of SOCSPOT with each plant functional group—as compared with consistency in the FAO GSOCSeq rates regardless of plant functional type—indicates variations due to above and belowground biomass are improved upon in SOCSPOT.

FIG. 11A is a comparison between the method results of the method 10 of the present invention and the prior art FAO GSOCSeq modeling method for the perennial forb/grass plant functional type as aligned with distributions from the literature. The distributions for the present invention for perennial forb/grass aligned with those from literature, where n=61. The present invention is shown at curve 126, the FAO model at curve 128 and the literature at curve 130.

FIG. 11B shows another comparison where the results of the method of the present invention and the prior art FAO GSOCSeq modeling method for the perennial tree functional type. The present invention is shown at curve, and the FAO model at curve 128.

FIG. 11C is a comparison between the results of the method of the present invention and the prior art FAO GSOCSeq modeling method for the perennial shrub plant functional type. The present invention is shown at curve 126, and the FAO model at curve 128.

FAO GSOCSeq method is driven by the RothC model developed for croplands and assume business as usual. As such, distributions FAO GSOCSeq 128 have similar means for all PFTs while the distributions of the present invention at 126 advantageously vary across each vegetation class (n=61) providing a more accurate result.

In accordance with the present invention 10, many advantages are provided over the prior art. For example, as to data integration & processing, it has been well known to use simple soil drainage classifications, static soil moisture assessments, single-point-in-time measurements, and separate processing of climate and soil data. However, the present invention 10 integrates a 30-year climate history with current satellite data, employs dynamic soil moisture response modeling with continuous temporal data processing at annual frequency as well as a unified processing framework for multiple data streams at 30 m-500 m resolution. A 30-years data history is preferred because this is the standard in climate science for a long-term condition, and as such, the datasets in the industry represent 30-years.

As to Microbial Response Modeling, it has been well known in the prior art to employ static microbial response curves with uniform treatment across climate regions, simple temperature-based activity models and binary moisture response thresholds. In contrast, the method and system 10 of the present invention uses climate-history-based microbial response curves, region-specific MMRC calculations, integrated MicrobialDegree Days (MDD) framework as well as continuous moisture response functions based on historical adaptation.

More specifically, the integration function and all the parameters that are used in the integration of the present invention is set forth below. A long-term 30 year averages of historical rainfall to establish what the typical ‘baseline’ moisture conditions are for a given land parcel. This is input into an equation (i.e., integration function, see below) that modifies how microbes will likely respond to contemporary soil moisture conditions (i.e., see below, mₒ) and the soil field capacity (i.e., see below, f). As such, this captures how historical moisture conditions will modulate the microbial response to present-day conditions, rather than assuming all communities respond in a 1-1 fashion to the same present-day moisture, which is known not to be true.

$$y = \frac{( \frac{1}{s \cdot \sqrt{3 π}} \cdot exp ( \frac{- \text{.75}}{\text{.025} {p}_{h}^{\text{.85}}} \cdot ( \frac{( x - {m}_{o )}}{\text{.25} {sf}^{\text{.25}}} ) \text{?}}{10}$$

$${m}_{o} = ( \frac{2 {p}_{h} \cdot ( 0.75 ) {f}^{\text{.35}}}{{p}_{mx}} ) = 0.69861708369$$

$$s = 1 \cdot \frac{( {p}_{mx} - {p}_{h} )}{{p}_{mx}} = 0.103571428571$$

$$y = \frac{( \frac{1}{s \cdot \sqrt{3 π}} \cdot exp ( \frac{- \text{.75}}{\text{.025} {p}_{h}^{\text{.85}}} \cdot {( \frac{( x - {m}_{o} )}{\text{.25} {sf}^{\text{.25}}} )}^{2} )}{10}$$

$${m}_{o} = ( \frac{2 {p}_{h} \cdot ( 0.75 ) {f}^{\text{.35}}}{{p}_{mx}} )$$

$$s = 1 \cdot \frac{( {p}_{mx} - {p}_{h} )}{{p}_{mx}}$$

$$\text{?} \text{indicates text missing or illegible when filed}$$

This integration function is what is used to develop the response curves which are broadly “region specific MMRC calculations” as discussed above. These are the continuous response functions that use current inputs from time series of satellite data for processing. This is generally referred to as (f) a dynamic soil moisture response model in accordance with the present invention.

With regard to carbon Dynamics, prior art methods use fixed sequestration rate estimates with a storage capacity focus, single vegetation type responses and binary saturation thresholds. On the other hand, the method and system 10 of the present invention employs dynamic sequestration rate modeling with integration of saturation effects on rates, plant Functional Type-specific baseline rates as well as continuous saturation adjustment functions. For example, as soil fills/saturates with carbon, they are reaching their capacity, and the rate of carbon assimilation will slow down. The present invention accounts for this effect using a saturation-adjustment equation that “slows” the sequestration rate as the soil approaches a “full” state.

Moreover, the present invention calculates carbon input baseline rates based on the combination of plant-functional type (PFT==what ‘class’ of vegetation it is; e.g., grass, shrub, tree, crop, and the like), the aboveground carbon, and the belowground carbon. All three of these factors are combined through the equations of the present invention to generate the ‘baseline rates’ for each PFT.

As to computational Frameworks, it is known that the prior art uses complex biogeochemical models with heavy computational requirements, fixed parameter relationships, and single-purpose outputs. However, the present invention greatly improves upon known prior art systems by employing a reduced complexity modeling framework with an efficient processing architecture, with dynamic parameter relationships and multi-attribute output generation (MMRC, MDD, sequestration potential.

The aforesaid examples are only one of the potentially many applications and modes of execution of the system of the present invention and common changes and substitutes made by technical personnel of this field within the technical proposal of this invention should be included in the protection scope thereof. It would be appreciated by those skilled in the art that various changes and modifications can be made to the illustrated embodiments without departing from the spirit of the present invention. All such modifications and changes are intended to be covered by the appended claims.

What is claimed is:

1. A method for predicting soil organic carbon dynamics, comprising the steps of:
providing readily available data streams selected from the group including satellite-based
(i) soil moisture,
(ii) vegetation measurements,
(iii) historical climate patterns,
predicting microbial response to environmental conditions based on the data streams;
generating high-resolution maps of sequestration potential and microbial conditions.

2. The method of claim 1, wherein the high-resolution maps are generated from information at high spatial resolution and annual temporal frequency.

3. The method of claim 2, wherein the high spatial resolution is 30 m to 500 m.

4. The method of claim 1, wherein the high-resolution maps of sequestration and microbial conditions are maps of relative sequestration potential across landscapes.

5. The method of claim 2, further comprising the step of rescaling the information to higher resolutions.

6. The method of claim 1, further comprising the step of:
providing integration of modern satellite data streams of soil moisture, vegetation measurements and/or historical climate patterns, to predict microbial response to environmental conditions.

7. The method of claim 1, further comprising the step of:
providing integration of multiple data streams with development of quantitative Microbial Moisture Response Coefficient, MicrobialDegree Days, Microbial Activity Responses to Soil pH and Carbon Use Efficiency metrics.

8. The method of claim 7, wherein the integration is a function of the following:
$$y = \frac{( \frac{1}{s \cdot \sqrt{3 π}} \cdot exp ( \frac{- \text{.75}}{\text{.025} {p}_{h}^{\text{.85}}} \cdot ( \frac{( x - {m}_{o} )}{\text{.25} {sf}^{\text{.25}}} ) \text{?}}{10}$$
$${m}_{o} = ( \frac{2 {p}_{h} \cdot ( 0.75 ) {f}^{\text{.35}}}{{p}_{mx}} ) = 0.69861708369$$
$$s = 1 \cdot \frac{( {p}_{mx} - {p}_{h} )}{{p}_{mx}} = 0.103571428571$$
$$\text{?} \text{indicates text missing or illegible when filed}$$
wherein y is the final predicted MMRC value, mₒ is the optimal moisture condition, s is the difference between the maximum precipitation (pₘₓ) and characteristic historical condition (pₕ), x is current soil moisture expressed as a fraction (0-1) f is field capacity to calculate a region-specific Microbial Moisture Response Coefficient.

9. The method of claim 7, wherein the Microbial Moisture Response Coefficient metric is derived from current satellite-derived soil moisture measurements of soil moisture, vegetation measurements and/or historical climate patterns, as well as soil physical structure and soil chemical properties, to predict microbial response to environmental conditions.

10. The method of claim 7, further comprising the steps of:
deriving the MicrobialDegree Days metric from transfer of GrowingDegree Days from agronomic forecasting to account for effects of soil temperature on microbial activity; wherein MicrobialDegree Days has normalized scores in the range of 0-10 with a formula of:
$$MDD = mean ( daily\_temp \_response \_factor )$$
adjusting sequestration potential predictions based on current carbon saturation levels.

11. The method of claim 10, wherein carbon dynamics is processed using a baseline carbon sequestration potential that is adjusted based on the saturation percentage of mineral-associated organic carbon content that is currently present.

12. The method of claim 7, wherein the MicrobialDegree Days is calculated by:
$$mean ( daily\_temp \_response \_factor ) .$$

13. The method of claim 7, further comprising the step of generating a dimensionless sequestration coefficient based on the product of Microbial Moisture Response Coefficient, MicrobialDegree Days, and soil pH scalar.

14. The method of claim 7, wherein the Microbial Moisture Response Coefficient ranges between 0 and 10.

15. The method of claim 13, wherein the soil pH scalar coefficient is determined by:
$$\frac{10}{( \sqrt{2 π {s}^{2}} )} exp ( - \frac{{( x - m )}^{2}}{12 {s}^{2}} )$$
wherein x reflects the soil pH value for a given location, s is a scalar value currently tuned and set to 0.4, and m is a scalar currently set to the pH that corresponds with peak microbial activity (i.e., 6), wherein the foregoing are variables configured and arranged to be adjusted values for a range of alternative conditions.

16. The method of claim 1, further comprising the step of:
providing integration of historical adaptation with microbial response modeling with saturation effects on rates and multi-attribute output generation.

17. The method of claim 16, wherein rate modeling is dynamic sequestration rate modeling.

18. The method of claim 7, wherein the Microbial Moisture Response Coefficient is calculated by:
$$f ( current\_moisture , historial\_climate , field\_capacity ) .$$

19. The method of claim 1, further comprising the step of:
continuously adjusting the microbial responses to contemporary conditions based on historical climate regimes.

20. The method of claim 1, wherein the historical climate patterns are thirty-year precipitation averages.

21. The method of claim 1, wherein the method accounts for how the historical climate patterns influence microbial community behavior to more accurately predict potential carbon sequestration rates across diverse landscapes.

22. The method of claim 20, further comprising the step of:
establishing a baseline metabolic activity response curve of the microbial community at a given location based on the thirty-year precipitation averages.

23. The method of claim 1, further comprising the step of:
using dynamic microbial response modeling.

24. The method of claim 1, further comprising the step of:
using gridded geospatial data results, producing estimates at global scale without prior farm-specific input data.

25. The method of claim 1, further comprising the step of:
providing an integration framework.

26. The method of claim 1, wherein:
$$a Final Sequestration Potential = baseline\_ad \_seq \_rate \times seq\_score \times saturation\_adjustment \_factor ;$$
$$a baseline\_ad \_seq \_rate = f ( biomass , CUE ) , where biomass = f ( NPP , land cover types ) ; and$$
$$a seq\_score = f ( MDD score , MpHC , MMRC score ) .$$

27. The method of claim 1, further comprising the step of:
providing a temporal analysis component.
